(19) United States
(12) Patent Application Publication
(10) Pub. No.: US 2003/0119188 A1
(43) Pub. Date: Jun. 26, 2003

(54) Novel compounds

(76) Inventors: Patrick Camilleri, Harlow (GB); Anthony John Kirby, Cambridge (GB); Christele Perrin, Cambridge (GB); Caroline McGregor, Cambridge (GB)

Correspondence Address: GLAXOSMITHKLINE, Corporate Intellectual Property - UW2220, P.O. Box 1539, King of Prussia, PA, 19406-0939, US

(21) Appl. No.: 10/119,598

(22) Filed: Apr. 10, 2002

(30) Foreign Application Priority Data
Oct. 11, 2001 (WO) PCT/GB01/04529

(51) Int. Cl. A61K 48/00; A61K 38/10; A61K 38/08; C07K 7/08; C07K 7/06; C12N 15/85

(52) U.S. Cl. 435/455; 514/17; 514/16; 530/328; 530/329; 514/44

(57) ABSTRACT

Peptide-based gemini compounds comprising basic amino acid chains linked by at least epsilon amide bond, showing improved DNA transfection properties, are disclosed. Methods for production of the compounds and the uses thereof are also disclosed.

### RELATED APPLICATIONS

This application claims the benefit of priority GB Patent Application No.: PCTIGB01/04529, filed Oct. 11, 2001; which claims the benefit of Great Britain application number GB 0025190.0, filed Oct. 12, 2000.

This invention relates to peptide-based gemini surfactant compounds, to the use of such compounds and to their production. The invention also relates to the use of the peptide-based gemini compounds to facilitate the transfer of compounds into cells for drug delivery.

Surfactants are substances that markedly affect the surface properties of a liquid, even at low concentrations. For example surfactants will significantly reduce surface tension when dissolved in water or aqueous solutions and will reduce interfacial tension between two liquids or a liquid and a solid. This property of surfactant molecules has been widely exploited in industry, particularly in the detergent and oil industries. In the 1970s a new class of surfactant molecule was reported, characterised by two hydrophobic chains with polar heads which are linked by a hydrophobic bridge (Deinega, Y et al., Kolloidn. Zh. 36, 649, 1974). These molecules, which have been termed “gemini” (Menger, F M and Littau, C A, J. Am. Chem. Soc. 113, 1451, 1991), have very desirable properties over their monomeric equivalents. For example they are highly effective in reducing interfacial tension between oil and water based liquids and have a very low critical micelle concentration.

Cationic surfactants have been used inter alia for the transfection of polynucleotides into cells in culture, and there are examples of such agents available commercially to scientists involved in genetic technologies (for example the reagent Tfx™-50 for the transfection of eukaryotic cells available from Promega Corp. WI, USA).

The efficient delivery of DNA to cells in vivo, either for gene therapy or for antisense therapy, has been a major goal for some years. Much attention has concentrated on the use of viruses as delivery vehicles, for example adenoviruses for epithelial cells in the respiratory tract with a view to corrective gene therapy for cystic fibrosis (CF). However, despite some evidence of successful gene transfer in CF patients, the adenovirus route remains problematic due to inflammatory side-effects and limited transient expression of the transferred gene. Several alternative methods for in vivo gene delivery have been investigated, including studies using cationic surfactants. Gao, X et al. (1995) Gene Ther. 2, 710-722 demonstrated the feasibility of this approach with a normal human gene for CF transmembrane conductance regulator (CFTR) into the respiratory epithelium of CF mice using amine carrying cationic lipids. This group followed up with a liposomal CF gene therapy trial which, although only partially successful, demonstrated the potential for this approach in humans (Caplen, N. J. et al., Nature Medicine, 1, 39-46, 1995). More recently other groups have investigated the potential of other cationic lipids for gene delivery, for example cholesterol derivatives (Oudrhiri, N et al. Proc. Natl. Acad. Sci. 94, 1651-1656, 1997). This limited study demonstrated the ability of these cholesterol based compounds to facilitate the transfer of genes into epithelial cells both in vitro and in vivo, thereby lending support to the validity of this general approach.

These studies, and others, show that in this new field of research there is a continuing need to develop novel low-toxicity surfactant molecules to facilitate the effective transfer of polynucleotides into cells both in vitro for transfection in cell-based experimentation and in vivo for gene therapy and antisense treatments. The present invention seeks to overcome the difficulties exhibited by existing compounds.

The invention relates to the peptide-based gemini compounds comprising two linked chains:

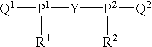

each chain having:

(1) a positively charged hydrophilic head, Q¹ or Q², formed from one or more amino acids and/or amines;

(2) a central portion, P¹ or P², having a polypeptide backbone; and

(3) a hydrophobic tail, R¹ or R²;

the central sections of each chain being linked together by bridge Y through residues in P¹ and P².

Preferably the central portion is made up of two or three amino acids, P^{a} (optional), P^{b} and P^{c}, in which:

P^{a} is a D- or L-amino acid, preferably hydrophilic, such as threonine or serine,

P^{b} is preferably D- or L-cysteine, serine or threonine, and

P^{c} is preferably D- or L-serine or threonine and is linked to R¹ or R².

Preferred compounds of the present invention include compounds of the formula (I):

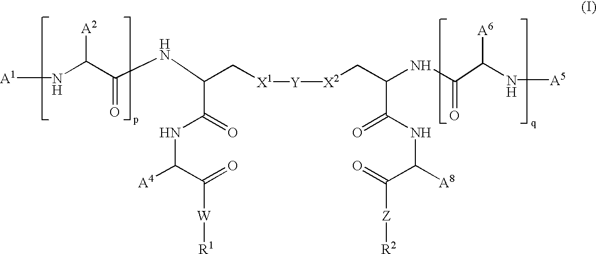

where:

A¹ and A⁵, which may be the same or different, is a positively charged group formed from two or more basic amino acids wherein the amide bonds linking said basic amino acids include at least one epsilon (ε) amide bond;

A²/A⁶CH(NH)CO, which may be the same or different, is derived from an amino acid, preferably serine;

p and q, which may be the same or different, is 0 or 1;

X¹/X²CH₂CH(NH)CO, which may be the same or different, is derived from cysteine (X¹/X²=S), serine or threonine (X¹/X²=O);

A⁴/A⁸CH(NH)CO, which may be the same or different, is derived from serine or threonine;

Y is a linker group, preferably (CH₂)ₘ where m is an integer from 1 to 6, most preferably 2, and may be a disulphide bond when X¹ and X² is each S;

R¹ and R² are C₍₁₀₋₂₀₎ saturated or unsaturated akyl groups, and

W and Z are NH, O, CH₂ or S; or

a salt, preferably a pharmaceutically acceptable salt thereof.

Preferably, the compound is symmmetrical, that is A¹ and A⁵ are the same, A² and A⁶ are the same, A⁴ and A⁸ are the same, R¹ and R² are the same, and W and Z are the same.

Representative examples of A¹/A⁵ include D- or L-amino acids selected from arginine, lysine, ornithine and histidine, preferably lysine, or amines such as spelyin and spermidine. Up to seven amino acids and thr amines may be linked in a linear or branched chain. Prefered examples include groups having two or three lysines or ornithines or a combination of aysine, ornithine, arginine and histidine, for instance:

COCH(NHR)(CR₂)₄NHCO(NH₂)(CH₂)₄NH₂

or

COCH(NHR)(CH₂)₃NHCO(NH₂)(CH₂)₃NH₂

or

COCH(NHR)(CH₂)₄NHCO(NH₂(CH₂)₃NH₂

in which R is H or NHCO(NH₂)(CH₂)₄NH₂ or NHCO(NH₂)(CH₂)₃NH₂

Preferably, —X¹—Y—X²— is —SCH₂CH₂S— or —OCH₂CH₂O—

Preferably, R¹ and R² is each a C₁₂-C₂₀ alkyl group, for instance C₁₂.

Preferably, W and Z is NH, thereby forming a further amide (CONH) bond.

Gemini compounds of formula (I) are disclosed in WO99/29712 (SmithKline Beecham). However it has been surprisingly found that compounds of formula (I) wherein the amide bonds linking the basic amino acids of groups A¹, A⁵, A² and A⁶ include at least one epsilon (e) amide bond, demonstrate far superior gene transfection properties compared with equivalent compounds wherein the basic amino acids are linked in a linear manner via α amide bonds.

“By means of epsilon (ε) amide bonds” is defined as meaning that the peptide bond is not created using the usual, α, amide group on the basic amino acid residue, but by means of the ε amide group at the end of the side chain of said basic amino acid. Such chains are therefore not “linear” in the sense of naturally bonded polypeptides, but rather are “branched”. In a preferred embodiment the basic amino acids are selected from lysine or arginine, most preferably lysines which are linked to each other by means of at least one, preferably two, epsilon (ε) amide bonds. Most preferably there are 3 lysines so linked (ie. p and q of formula (I) are both 0 and A¹ and A⁵ are -Lys-ε-Lys-ε-Lys).

Compounds of the present invention may be prepared from readily available starting materials using synthetic peptide chemistry well known to the skilled person. Such compounds may be synthesised, for example, starting with the construction of the di-cysteine part and subsequently building up the hydrophilic head by attaching a serine moiety at the carboxyl group of each cysteine moiety, using standard peptide chemistry, and then attaching the hydrocarbon chains to the carboxyl group of the serine moiety using a standard amide forming reaction well known to those skilled in the art. This intermediate can then be taken through to compounds of formula (I) by further reaction at the nitrogens of the cysteine residues.

Another aspect of the invention relates to methods for using the peptide-based gemini compounds. Such uses include facilitating the transfer of oligonucleotides and polynucleotides into cells for antisense, gene therapy and genetic immunisation (for the generation of antibodies) in whole organisms. Other uses include employing the compounds of the invention to facilitate the transfection of polynucleotides into cells in culture when such transfer is required, in, for example, gene expression studies and antisense control experiments among others. The polynucleotides can be mixed with the compounds, added to the cells and incubated to allow polynucleotide uptake. After further incubation the cells can be assayed for the phenotypic trait afforded by the transfected DNA, or the levels of mRNA expressed from said DNA can be determined by Northern blotting or by using PCR-based quantitation methods for example the Taqman® method (Perkin Elmer, Conn., USA). Compounds of the invention offer a significant improvement, typically between 3 and 6 fold, in the efficiency of cellular uptake of DNA in cells in culture, compared with compounds in the previous art. In the transfection protocol, the gemini compound may be used in combination with one or more supplements to increase the efficiency of transfection. Such supplements may be selected from, for example:

(i) a neutral carrier, for example dioleyl phosphatidylethanolamine (DOPE) (Farhood, H., et al (1985) Biochim. Biophys. Acta 1235 289);

(ii) a complexing reagent, for example the commercially available PLUS reagent (Life Technologies Inc. Maryland, USA) or peptides, such as polylysine or polyornithine peptides or peptides comprising primarily, but not exclusively, basic amino acids such as lysine, ornithine and/or arginine. The list above is not intended to be exhaustive and other supplements that increase the efficiency of transfection are taken to fall within the scope of the invention.

In still another aspect, the invention relates to the transfer of genetic material in gene therapy using the compounds of the invention.

Yet another aspect of the invention relates to methods to effect the delivery of non-nucleotide based drug compounds into cells in vitro and in vivo using the compounds of the invention.

The following definitions are provided to facilitate understanding of certain terms used frequently herein.

“Amino acid” refers to dipolar ions (zwitterions) of the form ⁺H₃NCH(R)CO₂⁻. They are differentiated by the nature of the group R, and when R is different from hydrogen can also be asymmetric, forming D and L families. There are 20 naturally occurring amino acids where the R group can be, for example, non-polar (e.g. alanine, leucine, phenylalanine) or polar (e.g. glutamic acid, histidine, arginine and lysine). In the case of un-natural amino acids R can be any other group which is not found in the amino acids found in nature.

“Polynucleotide” generally refers to any polyribonucleotide or polydeoxribonucleotide, which may be unmodified RNA or DNA or modified RNA or DNA. “Polynucleotides” include, without limitation single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, “polynucleotide” refers to triple-stranded regions comprising RNA or DNA or both RNA and DNA. The term polynucleotide also includes DNAs or RNAs containing one or more modified bases and DNAs or RNAs with backbones modified for stability or for other reasons. “Modified” bases include, for example, tritylated bases and unusual bases such as inosine. A variety of modifications have been made to DNA and RNA; thus, “polynucleotide” embraces chemically, enzymatically or metabolically modified forms of polynucleotides as typically found in nature, as well as the chemical forms of DNA and RNA characteristic of viruses and cells. “Polynucleotide” also embraces relatively short polynucleotides, often referred to as oligonucleotides.

“Transfection” refers to the introduction of polynucleotides into cells in culture using methods involving the modification of the cell membrane either by chemical or physical means. Such methods are described in, for example, Sambrook et al., MOLECULAR CLONING: A LABORATORY MANUAL, 2nd Ed., Cold Spring Harbor Laboratory Press, Cold Spring Harbor, N.Y. (1989). The polynucleotides may be linear or circular, single-stranded or double-stranded and may include elements controlling replication of the polynucleotide or expression of homologous or heterologous genes which may comprise part of the polynucleotide.

The invention will now be described by way of the following examples.

### EXAMPLE 1

### General Route for the Synthesis of Gemini Compounds

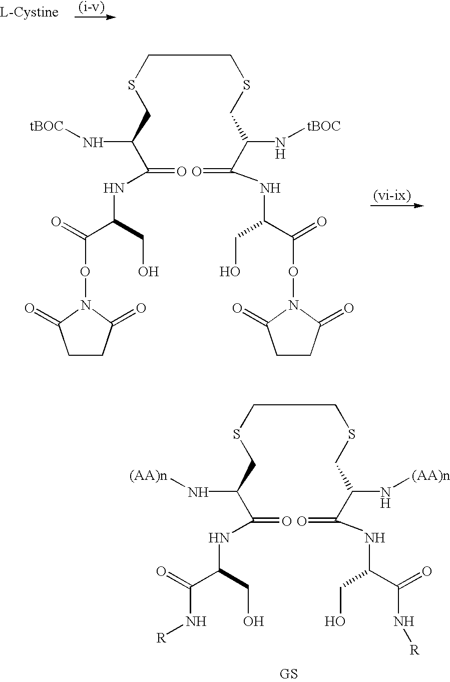

Conditions:

(i) BrCH₂CH₂Br, NaHCO₃, 88% yield;

(ii) Di-t-butyldicarbonate, NaOH, 66% yield;

(iii) N-hydroxysuccinimide, Dicyclohexylcarbodiimide, quantitative;

(iv) L-serine, K₂CO₃, 90% yield;

(v) N-hydroxysuccinimide, Dicyclohexylcarbodiimide, quantitative.

(vi) RNH₂ (dodecylamine, tetradecylamine, hexadecylamine, natural oleylamine (85/15 cis/trans), oleylamine and elaidylamine), triethylamine, 50-90% yield;

(vii) c.HCl, CHCl₃, 70-90% yield;

(viii) N-hydroxy-succinimide ester of BOC protected peptide (n.b. all peptides were prepared by traditional methods of peptide bond formation, NaOH, 50-70% yield;

(ix) c.HCl, CH₃OH, 50-70% yield.

### EXAMPLE 2

### Synthesis of Gemini Compounds

Gemini compounds of formula (I) wherein:

p and q are 0;

—X¹—Y—X²— is —SCH₂CH₂S—;

A¹ and A⁵ groups are as shown in Table 1;

R¹/R² groups (which are the same) are as shown in Table 1 (as R);

A⁴/A⁸ CH(NH)CO is derived from serine (ie. A⁴/A⁸ are each CH₂OH);

and W and Z are both NH

were synthesised according to the scheme shown in Example 1

TABLE 1
Structures of selected peptide-based Gemini surfactants C_{18:1}^{Δ9}
indicates that the 9th bond in the C₁₈ chain is a double bond.

| GS | A¹ and A⁵ | R |
|---|---|---|
| 1 | (Lys)₂Lys- | C₁₂ |
| 2 | Lys-ε-Lys-ε-Lys- | C₁₂ |
| 3 | Lys-ε-Lys-α-Lys- | C₁₂ |
| 4 | Lys-α-Lys-ε-Lys- | C₁₂ |
| 5 | Lys-ε-Lys-ε-Lys- | C₁₄ |
| 6 | Lys-α-Lys-α-Lys- | C₁₄ |
| 7 | Lys-ε-Lys-ε-Lys- | C₁₆ |
| 8 | Lys-α-Lys-α-Lys- | C₁₆ |
| 9 | Lys-α-Lys-ε-Lys- | C_{18:1}^{Δ9} |
| 10 | (Lys)₂Lys- | C_{18:1}^{Δ9} |
| 11 | Lys-ε-Lys-ε-Lys- | C_{18:1}^{Δ9} |
| 12 | Lys-α-Lys-α-Lys- | C_{18:1}^{Δ9} |
| 13 | Lys-ε-Lys-α-Lys- | C_{18 1}^{Δ9} |

### EXAMPLE 3

### Transfection of Luciferase Reporter Gene into CHO Cells Using Gemini Surfactants

The capabilities of the Gemini surfactants in Table 1 to mediate the transfer of a luciferase reporter gene across Chinese hamster ovary (CHO-DG44) cell membranes were compared to that of LipofectAMINE 200™ (L2000), a potent non-viral vehicle commercialised by Life Technologies. Transfection activity was determined by assay for luciferase activity.

CHO-DG44 cells were incubated with DNA at 5 concentrations (4, 8, 10, 20 and 30 uM) of gemini surfactants (GS). Luciferase activity (in counts per second(cps)) was averaged over 4 measurements. The full set of data over all 5 concentrations is shown for GS11 in Table 2. All other gemini surfactants are shown at 4 uM.

TABLE 2
Results for luciferase transfection experiments

| Compound | Luciferase (cps) |
|---|---|
| L2000 | 9 × 10⁵ |
| GS10 | 4 × 10⁴ |
| GS2 | 6 × 10⁴ |
| GS12 | 1.8 × 10⁵ |
| GS13 | 1.9 × 10⁵ |
| GS5 | 2.7 × 10⁵ |
| GS9 | 1.05 × 10⁶ |
| GS7 | 1.07 × 10⁶ |
| GS11 (4 uM) | 2.15 × 10⁶ |
| GS11 (8 uM) | 1.45 × 10⁶ |
| GS11 (10 uM) | 7.5 × 10⁵ |
| GS11 (20 uM) | 4 × 10⁴ |
| GS11 (30 uM | 1 × 10⁴ |

The results showed that increasing the length of the hydrocarbon ‘tail’ led to a substantial increase in transfection activity: single-chain analogs based on S-methyl cysteine were inactive (data not shown). Gene expression was also found to highly dependent on the nature of the amide linkages between the three lysine residues in the head-group. Thus three lysines linked through their ε-amino group rather than partial or total α-linkage provided optimal electrostatic interaction of these gemini compounds with DNA (Table 2).

### EXAMPLE 4

### Effect of Basic Polypeptides on Transfection Efficiency

The transfection efficiency of GS11 was investigated further using a luciferase reporter gene complexed with either PLUS reagent (Life Technologies) or poly-D,L-lysine (molecular weight range 1000-4000) prior to the addition of GS11. The cells were C2C 12 mouse muscle cells. The results are shown in Table 3.

TABLE 3
Results for basic polypeptide experiments

| Compounds added | Luciferase (cps) |
|---|---|
| None (background) | Not detectable |
| L2000 | 7 × 10⁵ |
| GS11 | 7 × 10⁵ |
| GS11 + PLUS reagent | 2.9 × 10⁶ |
| GS11 + polylysine (hmwt) | 4.1 × 10⁶ |
| GS11 + polylysine (lmwt) | 5.4 × 10⁶ |

The gene expression efficiencies of all the gemini surfactants in Table 1 are increased at least two-fold in the presence of the PLUS reagent, a basic polypeptide available commercially from Life Technologies. Replacement of the PLUS reagent by poly-lysine had the same enhancing effect, as shown in Table 3, which compares the transfection efficiencies of GS11 in C2C12 mouse muscle cells in the presence of several additives. The addition of either pure enantiomeric form of poly-lysine (molecular weight range 1000 to 4000) to the plasmid had the same result as the racemic mixture: the addition of poly-lysine of a higher molecular weight range produced a much smaller effect on the gene transfection efficiencies of the gemini compounds.

The gemini compounds of Table 1 are found to give good gene transfection efficiency in a variety of eukaryotic cells, besides CHO-DG44 and C2C12. These compounds are effective also with HEK 293 and cell lines of neuronal origin, such as SHSY-5Y and C6-15, normally considered difficult to transfect.

### EXAMPLE 5

### Lipoplex Studies

Preliminary studies on the formulation of gemini compounds of Table 1 in lipoplex systems have been carried out. The design of an appropriate lipoplex formulation can have substantial advantages for a chosen route of administration, as lipoplexes can preserve plasmid DNA structural integrity and can potentially achieve targetted delivery. In this experiment GS11 was combined with DOPE, a ‘helper lipid’, to form a multilamellar lipid vesicle suspension in aqueous medium. (The optical properties of this suspension were typical of those observed with multilamellar vesicles.) The vesicles formed were found by extrusion methodology to be less than one micron in diameter suggesting that a combination of gemini surfactants with DOPE should allow the preparation of liposomes of various sizes sizes and lipid compositions. These colloidal changes could be as significant as the changes in the molecular composition of the gemini surfactants in securing optimum gene expression in animal models.

All publications and references, including but not limited to patents and patent applications, cited in this specification are herein incorporated by reference in their entirety as if each individual publication or reference were specifically and individually indicated to be incorporated by reference herein as being fully set forth. Any patent application to which this application claims priority is also incorporated by reference herein in its entirety in the manner described above for publications and references.

1. A peptide-based gemini compound according to the formula (I): 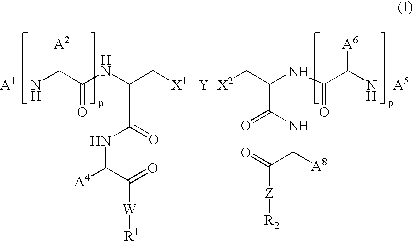
where:
A¹ and A⁵, which may be the same or different, is a positively charged group formed from two or more basic amino acids wherein the amide bonds linking said basic amino acids include at least one epsilon (ε) amide bond;
A²/A⁶CH(NH)CO, which may be the same or different, is derived from an amino acid;
p and q, which may be the same or different, is 0 or 1;
X¹/X²CH₂CH(NH)CO, which may be the same or different, is derived from cysteine (X¹/X²=S), serine or threonine (X¹/X²=);
A⁴/A⁸CH(NH)CO, which may be the same or different, is derived from serine or threonine;
Y is a linker group or a disulphide bond when X¹ and X² is each S;
R¹ and R² are C₍₁₀₋₂₀₎ saturated or unsaturated alkyl groups, and
W and Z are NH, O, CH₂ or S; or
a salt thereof.

2. A peptide-based gemini compound according to claim 1 selected from the table:

| A¹ and A⁵ | R |
|---|---|
| Lys-ε-Lys-ε-Lys- | C₁₂ |
| Lys-ε-Lys-α-Lys- | C₁₂ |
| Lys-α-Lys-ε-Lys- | C₁₂ |
| Lys-ε-Lys-ε-Lys- | C₁₄ |
| Lys-ε-Lys-ε-Lys- | C₁₆ |
| Lys-α-Lys-ε-Lys- | C_{18:1}^{Δ9} |
| Lys-ε-Lys-ε-Lys- | C_{18:1}^{Δ9} |
| Lys-ε-Lys-α-Lys- | C_{18 1}^{Δ9} |

wherein R refers to R¹ and R² which are the same.

3. A peptide-based gemini compound according to claim 2 wherein:
p and q are 0;
—X¹—Y—X²— is —SCH₂CH₂S—;
A⁴/A⁸ CH(NH)CO is derived from serine;
and W and Z are both NH.

4. The use of a gemini-based peptide compound as defined in any one of claims 1 to 3 in enabling transfection of DNA or RNA or analogs thereof into a eukaryotic or prokaryotic cell in vivo or in vitro.

5. The use of a peptide-based gemini compound according to claim 4 wherein the compound is used in combination with one or more supplements selected from the group consisting of:
(i) a neutral carrier; or
(ii) a complexing reagent.

6. The use according to claim 5 wherein the neutral carrier is dioleyl phosphatidylethanolamine (DOPE).

7. The use according to claim 5 wherein the complexing reagent is PLUS reagent.

8. The use according to claim 5 wherein the complexing reagent is a peptide comprising mainly basic amino acids.

9. The use according to claim 8 wherein the peptide consists of basic amino acids.

10. The use according to claim 8 or 9 wherein the basic amino acids are selected from lysine and arginine.

11. The use according to claim 10 wherein the peptide is poly-D,L-lysine with a molecular weight range of 1000 to 4000.

12. A method of transfecting polynucleotides into cells in vivo for gene therapy, which method comprises administering peptide-based gemini compounds of any one of claims 1 to 3 together with, or separately from, the gene therapy vector.

13. The use of a peptide-based gemini compound of any one of claims 1 to 3 to facilitate the transfer of a polynucleotide or an anti-infective compounds into prokaryotic or eukaryotic organism for use in anti-infective therapy.
